(19) United States
(12) Patent Application Publication
(10) Pub. No.: US 2012/0004487 A1
(43) Pub. Date: Jan. 5, 2012

(54) CATALYST FOR PRODUCING PARA-SUBSTITUTED AROMATIC HYDROCARBON AND METHOD FOR PRODUCING PARA-SUBSTITUTED AROMATIC HYDROCARBON USING THE SAME

(75) Inventors: Naoharu Igarashi, Toda-shi (JP); Chikanori Nakaoka, Toda-shi (JP)

(73) Assignee: JX NIPPON OIL & ENERGY CORPORATION, Tokyo (JP)

(21) Appl. No.: 13/256,060

(22) PCT Filed: Mar. 11, 2010
(86) PCT No.: PCT/JP2010/054615

§ 371 Date: Sep. 12, 2011

(30) Foreign Application Priority Data
Mar. 16, 2009 (JP) 2009-063092

(51) Int. Cl. C07C 2/66; B01J 29/70

(52) U.S. Cl. 585/467; 502/69

(57) ABSTRACT

This invention relates to a novel catalyst which enables an efficient production of a high-purity para-substituted aromatic hydrocarbon even without conducting isomerization step and/or adsorption separation step, and more particularly to a catalyst for producing a para-substituted aromatic hydrocarbon, which is formed by coating an MFI-type zeolite having an SiO₂/Al₂O₃ ratio (molar ratio) of 20 to 5000 and a primary particle size of not more than 1 μm with a crystalline silicate and is characterized by having a pKa value as measured by a Hammett indicator of not less than −8.

### TECHNICAL FIELD

This invention relates to a catalyst for producing a para-substituted aromatic hydrocarbon and a method for producing a para-substituted aromatic hydrocarbon using the catalyst, and more particularly to a catalyst which enables an efficient production of a high-purity para-substituted aromatic hydrocarbon.

### BACKGROUND ART

Among aromatic compounds, xylenes are very important compounds as a starting material for producing terephthalic acid, isophthalic acid, orthophthalic acid and so on for the formation of polyesters. These xylenes are produced, for example, by transalkylation, disproportionation or the like of toluene. However, p-xylene, o-xylene and m-xylene are existent in the resulting product as a structural isomer. Terephthalic acid obtained by oxidation of p-xylene is used as a main material for polyethylene terephthalate, and phthalic anhydride obtained from o-xylene is used as a starting material for plasticizer and the like, and isophthalic acid obtained from m-xylene is used as a main material for unsaturated polyesters and the like. Therefore, it is demanded to develop a method of separating these structural isomers from the product efficiently.

However, there is a little difference in the boiling point among p-xylene (boiling point: 138° C.), o-xylene (boiling point: 144° C.) and m-xylene (boiling point: 139° C.), so that it is difficult to separate these isomers by the usual distillation method. As the method of separating these isomers, there are a crystallization separation method wherein xylene mixture including p, o- and m-isomers is subjected to a precision distillation and thereafter p-xylene having a high melting point is separated by crystallization through cooling, a method wherein p-xylene is separated by adsorption with a zeolite-based adsorbent having a molecular sieving action, and so on.

In the method of selectively separating p-xylene by the crystallization separation, the crystallization through cooling should be conducted after the precision distillation of the xylene mixture including the structural isomers, so that there are problems that steps become multi-stages and complicated and the precision distillation and crystallization step through cooling cause the increase of production cost, and so on. For this end, the adsorption separation method is widely performed instead of the above method at the present. The latter method is a system in which the starting xylene mixture is moved through an adsorption tower filled with an adsorbent, during which p-xylene having a stronger adsorption force than those of the other isomers is adsorbed and separated from the other isomers. Subsequently, p-xylene is drawn out from the system through a desorbing agent and desorbed and separated from the desorbed liquid by distillation. As a practical process are mentioned PAREX method by UOP, AROMAX method by Toray Industries, Ltd. and so on. This adsorption separation method is high in the recovery and purity of p-xylene as compared with the other separation methods, but it is required to separate and remove the desorbing agent for removing p-xylene from the adsorbent since the adsorption and desorption are sequentially repeated in the adsorption tower comprising pseudo-moving beds of 10 to 20-odd stages, and hence the operation efficiency is never good in the high purification of p-xylene.

On the other hand, there are some attempts for improving the efficiency of the adsorption separation method for p-xylene, and also a method of conducting the separation while reacting with a catalyst having a separation function is disclosed. For example, Patent Document 1 mentioned below discloses a zeolite-combined zeolite catalyst comprising a first zeolite crystal with a catalytic activity and a second zeolite crystal with a molecular sieving action. In the zeolite-combined zeolite catalyst disclosed in Patent Document 1, however, the second zeolite crystal with the molecular sieving action forms a continuous phase matrix or bridge and the ratio of the first zeolite crystal with the catalytic activity occupied in the zeolite-combined zeolite catalyst becomes small and hence the deterioration of the catalytic activity is caused, but also when the second zeolite crystal with the molecular sieving action forms the continuous phase matrix, the permeation resistance of the molecule selected becomes too large, and it tends to deteriorate the molecular sieving action. Further, the second zeolite crystal plays the role of a binder (support) without using a binder (support) for shape holding, so that the zeolite-combined zeolite catalyst is obtained by aggregating or clumping the first zeolite crystal with the second zeolite crystal once. It is assumed that the aggregated or clumped catalyst needs to be shaped or granulated in use, but the second zeolite crystal is peeled off by the shear-fracture to expose a part of the first zeolite crystal, which causes the deterioration of the molecular sieving action.

Also, Patent Document 2 mentioned below discloses a method of coating solid acid catalyst particles with zeolite crystal having a molecular sieving action. In this method, however, an average particle size of the catalyst particles is as large as 0.3-3.0 mm and a thickness of the coating layer is as thick as 1-100 μm, and therefore the resistance of the body to be treated such as the starting materials, the products, or the like passing through the coating layer is large, so that it is assumed that a reaction efficiency is deteriorated, a conversion of toluene is low, a yield of paraxylene becomes notably low. On the other hand, as the thickness of the coating layer is thin, the coating layer might be easily damaged by physical stress, shear force or the like.

### PRIOR ART DOCUMENTS

Patent Documents

Patent Document 1: JP-A-2001-504084

Patent Document 2: JP-A-2003-62466

### SUMMARY OF THE INVENTION

Problems to be Solved by the Invention

As mentioned above, the conventional techniques cannot efficiently produce a high-purity para-substituted aromatic hydrocarbon, in particular, paraxylene, without complicated steps such as isomerization step and/or adsorption separation step.

It is, therefore, an object of the invention to solve the above-mentioned problems of the conventional techniques and to provide a novel catalyst which enables an efficient production of a high-purity para-substituted aromatic hydrocarbon even without conducting isomerization step and/or adsorption separation step, as well as a method for producing a high-purity para-substituted aromatic hydrocarbon using the catalyst.

Means for Solving Problems

The inventors have made various studies in order to achieve the above objects and discovered that a selectivity of an isomer having a specified structure can be improved to efficiently produce a high-purity para-substituted aromatic hydrocarbon, especially paraxylene, by using a catalyst formed by coating a specified MFI-type zeolite with a crystalline silicate and having a pKa value as measured by a Hammett indicator of not less than a certain value, because only the isomer having the specified structure among products selectively passes through a crystalline silicate film having a molecular sieving action inside the catalyst particles and conversely only the isomer having the specified structure selectively penetrates into the inside of the catalyst particles having a catalyst activity to cause a selective (specific) reaction inside the catalyst particles, and as a result the invention has been accomplished.

That is, the catalyst for producing a para-substituted aromatic hydrocarbon according to the invention is a catalyst formed by coating an MFI-type zeolite having an SiO₂/Al₂O₃ ratio (molar ratio) of 20 to 5000 and a primary particle size of not more than 1 μm with a crystalline silicate and characterized by having a pKa value as measured by a Hammett indicator of not less than −8.2.

In a preferable embodiment of the catalyst for producing a para-substituted aromatic hydrocarbon according to the invention, the crystalline silicate is a silicalite.

Also, the method for producing a para-substituted aromatic hydrocarbon according to the invention is characterized in that the para-substituted aromatic hydrocarbon is produced from an aromatic hydrocarbon in the presence of the above-described catalyst.

### EFFECT OF THE INVENTION

The catalyst according to the invention can be preferably used in the selective production of the para-substituted aromatic hydrocarbon by utilizing a molecular sieving action of the MFI-type zeolite, because the outer surface of the MFI-type zeolite is coated with the inert crystalline silicate film. In particular, a reaction on an outer surface having no selectivity of a catalyst can be suppressed by coating a ZSM-5 having MFI structure with a crystalline silicalite film having the similar structure, whereby the shape selectivity of paraxylene can be given to the catalyst and there can be provided an excellent catalyst for producing an industrially useful paraxylene selectively.

### BRIEF DESCRIPTION OF THE DRAWINGS

FIG. 1 shows a TEM photograph of a catalyst F.

### MODES FOR CARRYING OUT THE INVENTION

[Catalyst for Producing Para-Substituted Aromatic Hydrocarbon]

The catalyst for producing the para-substituted aromatic hydrocarbon according to the invention is formed by coating an MFI-type zeolite having an SiO₂/Al₂O₃ ratio (molar ratio) of 20 to 5000 and a primary particle size of not more than 1 μm with a crystalline silicate and is characterized by having a pKa value as measured by a Hammett indicator of not less than −8.2.

The zeolite with the MFI structure used as a nucleus for the catalyst according to the invention exhibits an excellent catalyst performance for producing para-substituted aromatic hydrocarbon structure-selectively through reaction between mutual aromatic hydrocarbons or between aromatic hydrocarbon and alkylating agent. As the MFI-type zeolite are preferably used various silicate materials such as ZSM-5, TS-1, TSZ, SSI-10, USC-4, NU-4 and so on. These zeolites can distinguish paraxylene from orthoxylene or metaxylene having a molecular size slightly larger than that of paraxylene because they have a pore size of 0.5-0.6 nm which is same as minor axis of paraxylene molecule, and are particularly effective in the case where the target para-substituted aromatic hydrocarbon is paraxylene.

The MFI-type zeolite as a nucleus for the catalyst has a primary particle size of not more than 1 μm. When the primary particle size of the MFI-type zeolite exceeds 1 μm, it cannot be used industrially because the reaction field required for the target reaction, i.e., a specific surface area of the catalyst is very small and thereby the reaction efficiency is deteriorated and also the diffusion resistance becomes larger and the conversion and the para-selectivity of the starting aromatic hydrocarbon become lower. Moreover, as the primary particle size of the MFI-type zeolite used is made smaller, the influence of diffusion inside the pores can be mitigated, so that it is preferably not more than 500 nm, more preferably not more than 200 nm, most preferably not more than 100 nm. The primary particle size of the MFI-type zeolite used can be measured by means of an X-ray diffractometer (XRD). In a method for measuring a particle size of the MFI-type zeolite, a particle size distribution analyzer, an electron scanning microscope (SEM) or the like may be sometimes used. However, in these methods, not a primary particle size but an agglomerated particle size (0.3 μm to 300 μm) derived from an agglomeration of many primary particle sizes may be sometimes measured. Therefore, when the primary particle size is measured by these methods, a concurrent use of the X-ray diffractometer (XRD) or the like is required for a confirmation.

Also, the SiO₂/Al₂O₃ ratio (molar ratio) of the MFI-type zeolite is 20 to 5000, preferably 25 to 1000, more preferably 30 to 300. When the SiO₂/Al₂O₃ ratio is less than 20, it is difficult to stably hold the MFT structure, while when it exceeds 5000, the amount of an acid being a reaction active site becomes undesirably small and the reaction activity is deteriorated.

The catalyst according to the invention is formed by coating the aforementioned MFI-type zeolite with a crystalline silicate, in which the crystalline silicate develops a molecular sieving action. The crystalline silicate film having the molecular sieving action (zeolite film) is preferable to have the structure similar to that of the-MFI type zeolite as the nucleus and to continue into the pores of the MFI-type zeolite. As a method for confirming the continuity of the pores are mentioned a method for measuring a diffusion rate of hydrocarbons having a different molecular size or determining whether or not they can penetrate, a method for measuring an increase of a crystallite diameter after coating by an X-ray diffraction, a method for observing a continuity of a lattice image at a junction part between the-MFI type zeolite and the crystalline silicate by means of a transmission electron microscope (TEM) and so on.

Further, the crystalline silicate is desirable to be inactive to disproportionation reaction and alkylation reaction, and is particularly preferable to be pure silica zeolite containing no alumina component (silicalite). Since silicalite is very few in the acid point, it is particularly preferable for inactivating the outer surface. Moreover, silicon in the crystalline silicate film may be partially replaced with another element such as gallium, germanium, phosphorus, boron or the like. Even in the latter case, it is important to maintain the surface in the inactivated state for a side reaction of the target reaction.

The amount of the crystalline silicate film by weight depends on the particle size of the MFI-type zeolite as a nucleus and is preferably not less than 1 part, more preferably not less than 5 parts but preferably not more than 100 parts, more preferably not more than 70 parts per 100 parts of the MFI-type zeolite as a nucleus. When the amount of the crystalline silicate is less than 1 part by weight per 100 parts by weight of the MFI-type zeolite, the molecular sieving action of the crystalline silicate film cannot be developed sufficiently, while when it exceeds 100 parts by weight, the ratio of the MFI-type zeolite in the catalyst becomes too small to cause the deterioration of the catalyst activity, but also the resistance of a body to be treated such as starting materials, products or the like passing through the crystalline silicate film may become too large. In this connection, the thickness of the crystalline silicate film is preferably not less than 1 nm, more preferably not less than 5 nm, but is preferably not more than 500 nm, more preferably not more than 100 nm. When the thickness of the crystalline silicate film is less than 1 nm, the molecular sieving action of the crystalline silicate film cannot be developed sufficiently, while when it exceeds 500 nm, the thickness of the crystalline silicate film is too thick and the resistance of the body to be treated such as starting materials, products or the like passing through the crystalline silicate film becomes too large.

In the invention, the method for coating the full surfaces of individual particles of the MFI-type zeolite with the crystalline silicate film is not particularly limited, but the conventional method for the preparation of zeolite film such as hydrothermal synthesis method or the like can be used. For example, a silica source such as formless silica, amorphous silica, fumed silica, colloidal silica or the like, a structure defining agent such as tetrapropylammonium hydroxide or the like and a minerallizer such as hydroxide of an alkali metal or an alkaline earth metal, and so on are first dissolved in water or ethanol in accordance with the composition of the target crystalline silicate film to prepare a sol for the formation of the crystalline silicate film.

Then, the individual particles of the MFI-type zeolite are immersed into the sol for the formation of the crystalline silicate film, or the sol for the formation of the crystalline silicate film is applied to the individual particles of the MFI-type zeolite, whereby the surfaces of the individual particles of the MFI-type zeolite are treated with the sol for the formation of the crystalline silicate film. Next, hydrothermal treatment is conducted to form a crystalline silicate film on each of the full surfaces of the individual particles of the MFI-type zeolite.

The hydrothermal treatment can be conducted by immersing the particles of the MFI-type zeolite treated with the sol for the formation of the crystalline silicate film into hot water or leaving them to stand in a heated steam. Concretely, the particles of the MFI-type zeolite may be heated in an autoclave while immersing them into the sol for the formation of the crystalline silicate film, or a heat-resistant and closed vessel including the particles of the MFI-type zeolite and the sol for the formation of the crystalline silicate film therein may be directly placed in an oven and then heated.

The hydrothermal treatment is carried out at a temperature of preferably not lower than 100° C. but not higher than 250° C., more preferably not lower than 120° C. but not higher than 200° C. for a time of preferably not less than 0.5 hour but not more than 72 hours, more preferably not less than 1 hour but not more than 48 hours. By conducting the hydrothermal synthesis, a crystal of silicate not having an active site can be epitaxially grown on a crystal of the MFI-type zeolite. In this regard, the epitaxial growth refers to crystal growth phenomenon in which one crystal grows on a surface of another crystal with a certain relation in crystal orientation, as shown in “Dictionary of Catalyst”, edited by Yoshio Ono, Makoto Mizono, Yoshihiko Morooka et al., secondary printed, Asakura Publishing Co., Ltd., p.110, 10th Apr. 2004. That is, the epitaxial growth in the present invention means a state where a crystalline silicate has the same structure as the MFI-type zeolite as a nucleus and forms a crystal phase continuing into a crystal phase of the nucleus and therefore the pores are continuing.

After the hydrothermal treatment, the particles of the MFI-type zeolite are taken out and dried and further subjected to a heat treatment, whereby the crystalline silicate film is calcined. The calcination may be carried out by raising the temperature at a temperature rising rate of 0.1 to 10° C./min, if necessary, and thereafter conducting the heat treatment at a temperature of 500 to 700° C. for 0.1 to 10 hours.

The catalyst according to the invention has a pKa value as measured by a Hammett indicator of not less than −8.2, preferably not less than −5.6, more preferably not less than −3.0, even more preferably not less than +1.5, but preferably less than +6.8, more preferably less than +4.8, even more preferably less than +4.0. When the pKa value of the catalyst is not less than −8.2, shape-selective reaction can be efficiently conducted. In this connection, the pKa value can be controlled by a thickness of the silica coating film or a state of the formed coating film, for example, conditions during a preparation of a catalyst, especially, amounts of a silica source and a structure defining agent charged for coating the MFI-type zeolite with the crystalline silicate through the hydrothermal synthesis, a treatment temperature and so on.

[Evaluation of Catalyst Performance by Measuring pKa Value with Hammett Indicator]

The catalyst used is formed by coating full surfaces of the individual particles of the MFI type zeolite with the crystalline silicate film and exhibits the certain pKa value as measured in dehydrated benzene by means of the Hammett indicator. The pKa value by the Hammett indicator is an indicator showing acid and base strengths, general explanation and measurement method are described in a book in detail. That is, a pKa value of +7.0 means neutrality, and the higher than +7.0 the pKa value is, the stronger the base strength is, and the lower than +7.0 the pKa value is, the stronger the acid strength is.

The specific measurement of the pKa value in the invention is carried out by adding 0.05 g of a catalyst to 5 ml of dehydrated benzene, adding a very slight amount of a Hammett indicator thereto, shaking a little and mixing them, and then observing a color change. The Hammett indicator used for measuring the pKa value in the invention includes 2,4-dinitrotoluene (pKa: −13.75), p-nitrotoluene (pKa: −11.35), anthraquinone (pKa: −8.2), benzalacetophenone (pKa: −5.6), dicinnamalacetone (pKa: −3.0), benzeneazodiphenylamine (pKa: +1.5), p-dimethylaminoazobenzene (pKa: +3.3), 4-(phenylazo)-1-naphthylamine (pKa: +4.0), methyl red (pKa: +4.8), neutral red (pKa: +6.8), and so on. In the invention, when a catalyst makes a color of a Hammett indicator having a pKa of X change and the catalyst is colored, it is concluded that a pKa value of the catalyst is less than X, while when the catalyst does not make a color of a Hammett indicator having a pKa of Y change, it is concluded that the pKa value of the catalyst is not less than Y. Thus, the pKa value as measured by the Hammett indicator of not less than −8.2 means that a color of anthraquinone (pKa: −8.2) is not changed.

A spectrophotometer may be used for a judging method of acid strength as mentioned above. Concretely, it is carried out by adding 0.25 g of a catalyst to 7 ml of a solution of a Hammett indicator in dehydrated benzene having a predetermined concentration (each concentration is shown in Table 1), and judging color change in a catalyst, i.e., coloring degree due to color change of the Hammett indicator, by means of the spectrophotometer. In this regard, values of the a* and b*-coordinates in a L*a*b* color appearance system defined by Japan Industrial Standard JIS Z 8729 are measured by the spectrophotometer to conduct an observation of the color change (coloring degree). The Hammett indicators used for measuring a pKa value in the invention are mentioned above. As a standard, if a color difference (Δa* or Δb*) between a catalyst and a high-purity silica (NIPGEL AZ-200 made by Tosoh Silica Corporation) not making a color of a Hammett indicator change becomes a value shown in Table 1 when they are added to each Hammett indicator solution shown in Table 1, it is concluded that the catalyst makes the color of the Hammett indicator change (the catalyst is colored). In this coloring judgment, when a catalyst makes a color of a Hammett indicator having a pKa of X change and the catalyst is colored, it is concluded that a pKa value of the catalyst is less than X, while when the catalyst does not make a color of a Hammett indicator having a pKa of Y change, it is concluded that the pKa value of the catalyst is not less than Y. Thus, the pKa value as measured by the Hammett indicator of not less than −8.2 means that a color of anthraquinone (pKa: −8.2) is not changed.

TABLE 1
Judging standard for Coloring by Hammett indicator

| | | | value |
|---|---|---|---|
| | | concentration | deemed |
| Name of Hammett indicator | pKa value | [g/l] | as coloring |
| p-nitrotoluene | −11.35 | 1.0 | Δb* ≧ 6 |
| anthraquinone | −8.2 | 1.0 | Δb* ≧ 3.5 |
| benzalacetophenone | −5.6 | 1.0 | Δb* ≧ 5 |
| dicinnamalacetone | −3.0 | 0.01 | Δa* ≧ 6 |
| benzeneazodiphenylamine | +1.5 | 0.001 | Δb* ≦ −6 |
| p-dimethylaminoazobenzene | +3.3 | 0.01 | Δa* ≧ 9 |

[Method for Producing Para-Substituted Aromatic Hydrocarbon]

In the method for producing the para-substituted aromatic hydrocarbon according to the invention, the para-substituted aromatic hydrocarbon is selectively produced in the presence of the aforementioned catalyst by the reaction between mutual aromatic hydrocarbons (disproportionation) or the reaction between aromatic hydrocarbon and alkylating agent (alkylation). The term “para-substituted aromatic hydrocarbon” used herein means an aromatic hydrocarbon having two alkyl substituents on its aromatic ring in which one of the substituents is located in a para site to the other substituent.

The starting aromatic hydrocarbon includes benzene as well as alkylbenzenes such as toluene, and the starting aromatic hydrocarbon may contain an aromatic hydrocarbon other than benzene and alkylbenzenes. In a particularly preferable embodiment of the invention, paraxylene is selectively produced by using a starting material containing benzene and/or toluene. However, when paraxylene is a target product, a starting material containing metaxylene, orthoxylene or ethylbenzene is not preferable.

As the alkylating agent used in the invention are mentioned methanol, dimethyl ether (DME), dimethyl carbonate, methyl acetate and the like. They may be commercially available, but methanol or dimethyl ether made from synthetic gas such as mixed gas of hydrogen and carbon monoxide, or dimethyl ether produced through dehydration reaction of methanol may be the starting material. Moreover, as a potential impurity existing in the aromatic hydrocarbon such as benzene and alkylbenzenes and the alkylating agent such as methanol and dimethyl ether are mentioned water, an olefin, a sulfur compound and a nitrogen compound, but they are desirable to be small.

The ratio of the alkylating agent to the aromatic hydrocarbon in the alkylation reaction is preferably 5/1 to 1/20, more preferably 2/1 to 1/10, most preferably 1/1 to 1/5 as a molar ratio of methyl group to the aromatic hydrocarbon. When the alkylating agent is extremely large as compared with the aromatic hydrocarbon, undesirable reaction between mutual alkylating agents is promoting, resulting in the possibility that coking is caused to deteriorate the catalyst. On the other hand, when the alkylating agent is extremely small as compared with the aromatic hydrocarbon, the conversion of the alkylation reaction to the aromatic hydrocarbon is notably deteriorated. Further, when toluene is used as the aromatic hydrocarbon, the disproportionation reaction between mutual toluenes becomes promoted.

The disproportionation reaction or alkylation reaction is desirable to be carried out by feeding the starting aromatic hydrocarbon at a liquid hourly space velocity (LHSV) of not less than 0.01 h⁻¹, more preferably not less than 0.1 h⁻¹ but not more than 20 h⁻¹, more preferably not more than 10 h⁻¹ to contact with the above catalyst. The conditions of the disproportionation reaction or alkylation reaction are not particularly limited, but the reaction temperature is preferably not lower than 200° C., more preferably not lower than 230° C., most preferably not lower than 250° C. but preferably not higher than 550° C., more preferably not higher than 530° C., most preferably not higher than 510° C., and the pressure is preferably not less than atmospheric pressure, more preferably not less than 0.1 MPaG, most preferably not less than 0.5 MPaG but preferably not more than 20 MPaG, more preferably not more than 10 MPaG, even more preferably not more than 5 MPaG.

In the disproportionation reaction or the alkylation reaction, an inert gas such as nitrogen or helium or hydrogen for suppressing the coking may be circulated or pressurized. Moreover, when the reaction temperature is too low, the activation of the aromatic hydrocarbon or alkylating agent or the like is insufficient, and hence the conversion of the starting aromatic hydrocarbon is low, while when the reaction temperature is too high, a lot of energy is consumed but also it tends to shorten the catalyst life.

When the alkylation reaction or the disproportionation reaction of the aromatic hydrocarbon proceeds in the presence of the catalyst, it is assumed to form a para-substituted aromatic hydrocarbon as a target product as well as an ortho-substituted aromatic hydrocarbon and a metha-substituted aromatic hydrocarbon as a structural isomer, a mono-substituted aromatic hydrocarbon in which the carbon number in the substituent is increased as compared with the starting aromatic hydrocarbon, an unreacted aromatic hydrocarbon, an aromatic hydrocarbon having 3 or more substituents associated with the proceeding of the alkylation, and light gas. Among them, it is preferable that the component ratio of the para-substituted aromatic hydrocarbon becomes higher. As an indication of para-selectivity in the reaction, when an aromatic hydrocarbon with a carbon number of 8 in the product is taken into account, the selectivity of paraxylene among aromatic hydrocarbons with a carbon number of 8 is preferably not less than 95 mol %, more preferably not less than 97.5 mol %, even more preferably not less than 99.5 mol %, particularly not less than 99.7 mol %, most preferably not less than 99.9 mol % at the first stage of the reaction.

The reaction product obtained in the invention may be separated and concentrated by the existing method, but it is possible to isolate the product only by a simple distillation process since a para-substituted aromatic hydrocarbon with an extremely high purity is obtained selectively in the invention. That is, it can be divided by the simple distillation into a fraction having a boiling point lower than that of the unreacted aromatic hydrocarbon, a high-purity para-substituted aromatic hydrocarbon and a fraction having a boiling point higher than that of the para-substituted aromatic hydrocarbon. When the amount of the fraction having a boiling point higher than that of the para-substituted aromatic hydrocarbon is extremely small, the high-purity para-substituted aromatic hydrocarbon can be isolated only by distilling off a light fraction. Moreover, the unreacted aromatic hydrocarbon may be recycled as a starting material.

### EXAMPLES

The following examples are given in illustration of the invention and are not intended as limitations thereof.

<Preparations of Catalysts>

(Preparation of Catalyst A)

A mixed solution A of 57.4 g of ion-exchanged water, 17.2 g of ethanol and 4.83 g of tetrapropylammonium hydroxide (TPAOH) is prepared. Then, the mixed solution is added with 20.4 g of tetraethyl orthosilicate (TEOS) and stirred for 30 minutes. To this mixed solution is added 10 g of NH₄-type ZSM-5 catalyst (SiO₂/Al₂O₃=30 (mol ratio), primary particle size; 50-60 nm (measured by X-ray diffractometer (XRD))), and hydrothermal synthesis is carried out by using an autoclave at 165° C. for 24 hours to conduct a coating treatment. After the hydrothermal synthesis, the catalyst is washed and collected by filtration, and dried at 90° C. Then, to the catalyst subjected to the coating treatment once are further added the mixed solution A and 20.4 g of TEOS, and hydrothermal synthesis is carried out in the same manner as described above to conduct a coating treatment. After the hydrothermal synthesis, the catalyst is washed and collected by filtration, then dried at 90° C. and calcined at 600° C. for 5 hours to obtain a catalyst A.

The catalyst A has a pKa value as measured by a Hammett indicator by visual observation of not less than −8.2 but less than −5.6 (expressed as −8.2 to −5.6), i.e., makes a color of an indicator having a pKa of −5.6 change but does not make a color of an indicator having a pKa of −8.2 change. Further, in a judgment by a spectrophotometer, when the catalyst A is immersed in the Hammett indicator having a pKa of −8.2, Δb* is −7 and thereby it is concluded that there is no coloring, while when it is immersed in the Hammett indicator having a pKa of −5.6, Δb* is 16 and thereby it is concluded that it is colored. This result indicates that the pKa value is not less than −8.2 but less than −5.6, and is identical to the results of the visual observation. Further, from confirmations by an X-ray diffractometer (XRD) and a transmission electron microscope (TEM), it is seen that the surface of the ZSM-5 catalyst is coated with a silicalite film.

Measuring conditions of the primary particle size by the X-ray diffractometer (XRD) are shown below.

Measuring apparatus: RAD-1C made by Rigaku Electric Corporation

X-ray source: Culoal (λ=0.15 nm)

Tube voltage: 30 kV

Tube current: 20 mA

Measuring conditions Scanning speed: 4°/min
Step width: 0.02°
Slit: DS=1.0°, RS=0.3 mm, SS=1.0°

Observing conditions of the catalyst by the transmission electron microscope (TEM)

Measuring apparatus; JEM-2100F made by JEOL Ltd.

Accelerating voltage: 200 kV

Measuring conditions of color by the spectrophotometer

Measuring apparatus: CM-600 made by Konica Minolta Sensing Inc.

Color appearance system: L*a*b*

Field of View: 10° view-field

Light source: D₆₅

Measurement diameter/Light diameter: φ8 m/φ11 mm

Processing mode of specular reflection: Specular Component Excluded

(Preparation of Catalyst B)

A catalyst B is obtained in the same manner as in the catalyst A except that the coating treatment condition in the hydrothermal synthesis is 175° C. The catalyst B has a pKa value as measured by a Hammett indicator of −5,6 to −3.0, i.e., makes a color of an indicator having a pKa of −3.0 change but does not make a color of an indicator having a pKa of −5.6 change. Further, in a judgment by a spectrophotometer, when the catalyst B is immersed in the Hammett indicator having a pKa of −5.6, Δb* is 0 and thereby it is concluded that there is no coloring, while when it is immersed in the Hammett indicator having a pKa of −3.0, Δa* is 24 and thereby it is concluded that it is colored. This result indicates that the pKa value is not less than −5.6 but less than −3.0, and is identical to the results of the visual observation. Further, from confirmations by the X-ray diffractometer (XRD) and the transmission electron microscope (TEM), it is seen that the surface of the ZSM-5 catalyst is coated with a silicalite film.

(Preparation of Catalyst C)

A catalyst C is obtained in the same manner as in the catalyst A except that the coating treatment condition in the hydrothermal synthesis is 180° C. The catalyst C has a pKa value as measured by a Hammett indicator of +1.5 to +3.3, i.e., makes a color of an indicator having a pKa of +3.3 change but does not make a color of an indicator having a pKa of +1.5 change. Further, in a judgment by a spectrophotometer, when the catalyst C is immersed in the Hammett indicator having a pKa of +1.5, Δb* is −4 and thereby it is concluded that there is no coloring, while when it is immersed in the Hammett indicator having a pKa of +3.3, Δa* is 11 and thereby it is concluded that it is colored. This result indicates that the pKa value is not less than +1.5 but less than +3.3, and is identical to the results of the visual observation. Further, from confirmations by the X-ray diffractometer (XRD) and the transmission electron microscope (TEM), it is seen that the surface of the ZSM-5 catalyst is coated with a silicalite film.

(Preparation of Catalyst D)

Also, a commercially available NH₄-type ZSM-5 used in the preparation of the catalyst A and not being subjected to a coating treatment is dried at 90° C. and then calcined at 600° C. for 5 hours to obtain a catalyst D. The catalyst D has a pKa value as measured by a Hammett indicator of −13.75 to −11.35, i.e., makes a color of an indicator having a pKa of −11.35 change but does not make a color of an indicator having a pKa of −13.75 change.

(Preparation of Catalyst E)

6.7 g of tetrapropylammonium bromide (TPABr) is weighed, thereto are added 95.0 g of ion-exchanged water, 0.94 g of sodium nitrate nonahydrate, 6.25 g of 4N sodium hydroxide in water, and 10.00 g of colloidal silica, and hydrothermal synthesis is carried out in an autoclave at 180° C. for 24 hours. The resulting product is washed, filtrated, dried at 90° C., and then calcined at 600° C. for 5 hours to obtain a ZSM-5 (silica/alumina ratio: 120, primary particle size: 50 nm) as an in-house product. This is referred to as a catalyst E. The catalyst E has a pKa value as measured by a Hammett indicator of −13.75 to −11.35, i.e., reacts with an indicator having a pKa of −11.35 but does not react with an indicator having a pKa of −13.75.

(Preparation of Catalyst F)

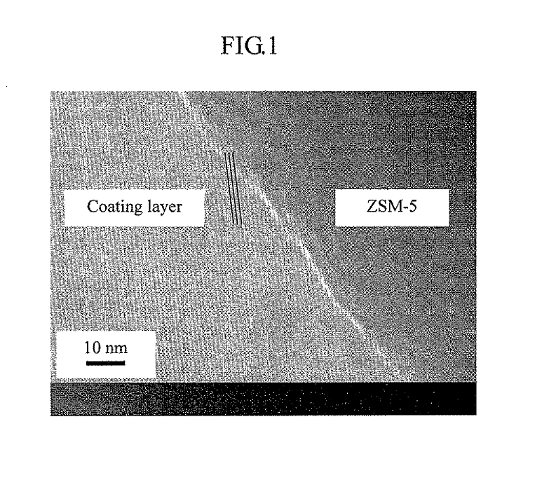

86.1 g of ion-exchanged water, 25,8 g of ethanol, 7.1 g of 10% tetrapropylammonium hydroxide (TPAOH) in water, and 30.6 g of tetraethyl orthosilicate (TEOS) are added and stirred for 30 minutes. To this mixed solution B is added 10 g of a commercially available ZSM-5 having a silica/alumina molar ratio of 300 and a primary particle size of 63 nm (measured by XRD), and hydrothermal synthesis is carried out in an autoclave at 180° C. for 24 hours to conduct a coating. The resulting product is washed, filtrated, dried, and then calcined at 600° C. for 5 hours to obtain a catalyst F. The catalyst F has a pKa value as measured by a Hammett indicator of −5.6 to −3.0, i.e., makes a color of an indicator having a pKa of −3.0 change but does not make a color of an indicator having a pKa of −5.6 change. In a judgment by a spectrophotometer, when the catalyst F is immersed in the Hammett indicator having a pKa of −5.6, Δb* is −5 and thereby it is concluded that there is no coloring, while when it is immersed in the Hammett indicator having a pKa of −3.0, Δa* is 20 and thereby it is concluded that it is colored. This result indicates that the pKa value is not less than −5.6 but less than −3.0, and is identical to the results of the visual observation. Further, from a confirmation by an XRD, it is seen that the resulting silicate has a MFI structure and a crystallite diameter increases to 67 nm. Further, from a observation of the ZSM-5 after the coating treatment by the TEM, it is seen that a lattice image of the ZSM-5 continues into that of the silicalite film, as shown in a TEM photograph of FIG. 1. Thus, it is seen that the silicalite film epitaxially grows on the surface of the ZSM-5 and coats it.

(Preparation of Catalyst G)

A commercially available ZSM-5 used in the preparation of the catalyst F and not being subjected to a coating treatment is calcined at 600° C. for 5 hours to obtain a catalyst G The catalyst G has a pKa value as measured by a Hammett indicator of −11.35 to −8.2, i.e., makes a color of an indicator having a pKa of −8.2 change but does not make a color of an indicator having a pKa of −11.35 change. In a judgment by a spectrophotometer, when the catalyst C is immersed in the Hammett indicator having a pKa of −11.35, Δb* is −8 and thereby it is concluded that there is no coloring, while when it is immersed in the Hammett indicator having a pKa of −8.2, Δb* is 14 and thereby it is concluded that it is colored. This result indicates that the pKa value is not less than −11.35 but less than −8.2, and is identical to the results of the visual observation.

(Preparation of Catalyst H)

To the mixed solution B is added 15.0 g of a commercially available ZSM-5 having a silica/alumina molar ratio of 30 and a primary particle size of 30-40 nm (measured by XRD), and hydrothermal synthesis is carried out in an autoclave at 180° C. for 24 hours to conduct a first coating. The resulting catalyst is washed, filtrated and dried. To this coated catalyst is added the mixed solution B again, and hydrothermal synthesis is carried out in an autoclave at 180° C. for 24 hours. After the hydrothermal synthesis, the catalyst is washed, collected by filtration, dried, and then calcined at 600° C. for 5 hours to obtain a catalyst H. The catalyst H has a pKa value as measured by a Hammett indicator of +1.5 to +3.3, i.e., makes a color of an indicator having a pKa of +3.3 change but does not make a color of an indicator having a pKa of +1.5 change. In a judgment by a spectrophotometer, when the catalyst C is immersed in the Hammett indicator having a pKa of +1.5, Δb* is −4 and thereby it is concluded that there is no coloring, while when it is immersed in the Hammett indicator having a pKa of +3.3, Δa* is 10 and thereby it is concluded that it is colored. This result indicates that the pKa value is not less than +1.5 but less than +3.3, and is identical to the results of the visual observation. Further, from confirmations by the X-ray diffractometer (XRD) and the transmission electron microscope (TEM), it is seen that the surface of the ZSM-5 catalyst is coated with a silicalite film.

Alkylation of toluene using ethanol as an alkylating agent>

### Example 1

An alkylation of toluene is carried out by diluting 0.05 g of a catalyst C with glass beads of 1.0 mmφ and filling them in a fixed layer reaction vessel of 4 mm in inner diameter to form a catalyst layer of 20 mm in length, and feeding toluene at a rate of 1.34 mmol/hr, methanol at a rate of 2.43 mmol/hr and helium gas at a rate of 22 ml/min, at 400° C. and an atmospheric pressure. After 1 hour from the start of the reaction, the product discharged from an outlet of the reaction vessel is analyzed by a gas chromatography to measure a ratio of each isomer in the product. The results are shown in Table 2, and the measuring conditions of the gas chromatography are shown below.

Measuring apparatus: GC-2014 made by Shimadzu Corporation

Column: capillary column Xylene Master made by Shinwa Chemical Industries Ltd., inner diameter of 0.32 mm, 50 m

Temperature condition: column temperature of 50° C., temperature rising rate of 2° C./min, temperature of detector (FID) of 250° C.

Carrier gas: helium

Toluene conversion (mol %)=100−(mol of residual toluene/mol of toluene in starting material)×100

Selectivity of paraxylene (mol %)=(mol of resulting paraxylene/mol of resulting C8 aromatic hydrocarbon)×100

### Comparative Example 1

A test is carried out in the same manner as in Example 1 except that the catalyst D is used.

TABLE 2

| | | | Comparative |
|---|---|---|---|
| | | Example 1 | Example 1 |
| Catalyst | | C | D |
| pKa | | +1.5 to +3.3 | −13.75 to −11.35 |
| Reaction temperature | ° C. | 400 | 400 |
| Alkylating agent | — | Methanol | Methanol |
| Toluene conversion | mol % | 2.0 | 66.0 |
| Paraxylene selectivity | mol % | >99.9 | 46.0 |
| Composition of product oil | | | |
| Benzene | mol % | <0.01 | <0.01 |
| Toluene | mol % | 98.00 | 34.00 |
| Ethylbenzene | mol % | <0.01 | <0.01 |
| Paraxylene | mol % | 2.00 | 23.00 |
| Methaxylene | mol % | <0.01 | 12.00 |
| Orthoxylene | mol % | <0.01 | 10.00 |
| Aromatic hydrocarbon having | mol % | <0.01 | 21.00 |
| a carbon number of 9 or more | | | |
| Total | mol % | 100.00 | 100.00 |

<Alkylation of Toluene Using Dimethyl Ether as an Alkylating Agent>

### Example 2

A test is carried out in the same manner as in Example 1 except that the catalyst A is used, dimethyl ether (DME) is used as an alkylating agent instead of methanol and a feed rate of the DME is 0.16 mmol/hr. The results are shown in Table 3.

### Example 3

A test is carried out in the same manner as in Example 2 except that the catalyst B is used and a reaction temperature is 350° C.

### Example 4

A test is carried out in the same manner as in Example 2 except that the catalyst C is used.

### Example 5

A test is carried out in the same manner as in Example 2 except that the catalyst F is added with silica (NIPGEL AZ-200 made by Tosoh Silica Corporation) as a binder, shaped (a mass ratio of catalyst F/binder=80/20), granulated at 16-24 mesh and filled in an amount of 0.06 g in a fixed layer reaction vessel having an inner diameter of 4 mm, and a reaction temperature is 350° C.

### Comparative Example 2

A test is carried out in the same manner as in Example 2 except that the catalyst E is used.

### Comparative Example 3

A test is carried out in the same manner as in Example 5 except that the catalyst G is used.

TABLE 3

| | | | | | | Comparative | Comparative |
|---|---|---|---|---|---|---|---|
| | | Example 2 | Example 3 | Example 4 | Example 5 | Example 2 | Example 3 |
| Catalyst | | A | B | C | F | E | G |
| pKa | | −8.2 to −5.6 | −5.6 to −3.0 | +1.5 to +3.3 | −5.6 to −3.0 | −13.75 to −11.35 | −11.35 to −8.2 |
| Reaction temperature | ° C. | 400 | 350 | 400 | 350 | 400 | 350 |
| Alkylating agent | — | DME | DME | DME | DME | DME | DME |
| Toluene conversion | mol % | 12.0 | 15.0 | 28.0 | 140 | 72.0 | 29.6 |
| Paraxylene selectivity | mol % | 96.0 | 98.0 | >99.9 | 99.7 | 51.0 | 74.8 |
| Composition of Product oil | | | | | | | |
| Benzene | mol % | <0.01 | <0.01 | 0.01 | <0.01 | <0.01 | <0.01 |
| Toluene | mol % | 88.00 | 85.00 | 72.00 | 85.98 | 28.00 | 70.40 |
| Ethylbenzene | mol % | <0.01 | <0.01 | <0.01 | <0.01 | <0.01 | <0.01 |
| Paraxylene | mol % | 12.00 | 11.00 | 28.00 | 12.98 | 24.00 | 21.04 |
| Methaxylene | mol % | 0.30 | <0.01 | <0.01 | <0.01 | 14.00 | 3.33 |
| Orthoxylene | mol % | 0.30 | 0.20 | <0.01 | 0.04 | 9.00 | 3.77 |
| Aromatic hydrocarbon having | mol % | 0.40 | 3.50 | <0.01 | 1.00 | 25.00 | 1.46 |
| a carbon number of 9 or more | | | | | | | |
| Total | mol % | 100.00 | 100.00 | 100.00 | 100.00 | 100.00 | 100.00 |

As seen from the Examples 1-5, a selectivity of p-xylene can be improved as high as not less than 96% as compared with a thermodynamic equilibrium composition (about 25%) by using methanol or DME as an alkylating agent and using the catalyst coated with a silicalite (catalyst A, B, C, F). In particular, it is seen that the selectivity of p-xylene is as very high as not less than 99.9% when the catalyst C is used.

<Disproportionation of Toluene>

### Example 6

The catalyst H is added with silica as a binder, shaped, granulated in the same manner as in Example 5 and then filled in an amount of 1.25 g in a fixed layer reaction vessel having an inner diameter of 10 mmφ. Then, a disproportionation of toluene is carried out at 400° C. and an atmospheric pressure under conditions that a ratio of hydrogen/toluene is 60 mol/mol and WHSV is 4.8 h⁻. The product discharged from an outlet of the reaction vessel is analyzed by a gas chromatography to measure a ratio of each isomer in the product. In this connection, the measuring conditions of the gas chromatography are the same as in Example 1. The results are shown in Table 4.

### Comparative Example 4

A test is carried out in the same manner as in Example 6 except that the catalyst D is used.

TABLE 4

| | | | Comparative |
|---|---|---|---|
| | | Example 6 | Example 4 |
| Catalyst | | H | D |
| pKa | | +1.5 to +3.3 | −13.75 to −11.35 |
| Reaction temperature | ° C. | 400 | 400 |
| Toluene conversion | mol % | 0.6 | 2.8 |
| Paraxylene selectivity | mol % | 96.4 | 26.5 |
| Paraxylene yield | mol % | 0.4 | 0.3 |

As seen from the Example 6, p-xylene is selectively produced by using a zeolite catalyst coated with a silicate (catalyst H) as a catalyst, so that the selectivity of p-xylene is as very high as 96.4% as compared with a thermodynamic equilibrium composition (about 25%). Also, the resulting oil contains substantially benzene (boiling point: 80° C.), paraxylene (boiling point: 138° C.) and aromatic hydrocarbons having a carbon number of not less than 9 (boiling point: 165 to 176° C.) in addition to toluene (boiling point: 110° C.) as a starting material, so that a high-concentration paraxylene can be easily obtained by distillation.

1. A catalyst for producing a para-substituted aromatic hydrocarbon, which is formed by coating an MFI-type zeolite having an SiO₂/Al₂O₃ ratio (molar ratio) of 20 to 5000 and a primary particle size of not more than 1 μm with a crystalline silicate and has a pKa value as measured by a Hammett indicator of not less than −8.2.

2. A catalyst for producing a para-substituted aromatic hydrocarbon according to claim 1, wherein the crystalline silicate is a silicalite.

3. A method for producing a para-substituted aromatic hydrocarbon, wherein the para-substituted aromatic hydrocarbon is produced from an aromatic hydrocarbon in the presence of a catalyst as claimed in claim 1.

4. A method for producing a para-substituted aromatic hydrocarbon, wherein the para-substituted aromatic hydrocarbon is produced from an aromatic hydrocarbon in the presence of a catalyst as claimed in claim 2.
